(19) United States
(12) Patent Application Publication
(10) Pub. No.: US 2025/0000427 A1
(43) Pub. Date: Jan. 2, 2025

(54) METHODS AND SYSTEMS FOR DETECTING AND ASSESSING COGNITIVE STATUS

(71) Applicant: SPARK NEURO INC., New York, NY (US)

(72) Inventors: Ché Lucero, Albuquerque, NM (US); Mackenzie Tweardy, New York, NY (US); Keith J. Yoder, Chicago, IL (US); Geoffrey Brookshire, Mill Valley, CA (US); Angelo Pennati, New York, NY (US); Spencer Gerrol, West Hartford, CT (US)

(21) Appl. No.: 18/758,754

(22) Filed: Jun. 28, 2024

Related U.S. Application Data
(60) Provisional application No. 63/524,073, filed on Jun. 29, 2023.

(51) Int. Cl. A61B 5/372; A61B 5/00

(52) U.S. Cl. CPC A61B 5/372; A61B 5/7203; A61B 5/725; A61B 5/726

(57) ABSTRACT

The present invention provides systems and methods for assessing the cognitive health of an individual.

### TECHNICAL FIELD

The present disclosure relates to methods and systems using neural data to assess neural cognition and pathologies.

### BACKGROUND

Neurological disorders are the leading cause of disability-adjusted life-years (sum of years of life lost and years lived with a disability) and the second leading cause of deaths. Alzheimer's disease (AD) and related dementias or (ADRD), Parkinson's disease and motor neuron diseases including amyotrophic lateral sclerosis, spinal muscular atrophy, hereditary spastic paraplegia, primary lateral sclerosis, progressive muscular atrophy, and pseudobulbar palsy collectively affected 4.7 to 6.0 million individuals in the U.S. between 2016-2017. By 2060, the prevalence ADRDs is expected to double. Accordingly, there has been intense interest in developing methods and systems that can accurately diagnose neurological disorders, assess their progression, and provide insight into appropriate treatment options.

With many developed countries facing increasingly aging populations, the prevalence of neurological and cognitive disorders has increased along with the needs to provide ongoing monitoring and diagnosis. Due to the complexity of the brain and human behavior, assessing cognitive/neural health presents a more intricate challenge than assessing conditions associated with known biomarkers, e.g., measures of blood pressure, hormones, and cholesterol. Simply put, there are few, non-invasive biomarkers known to have any assured association with cognitive impairment and progression of an underlying condition.

Consequently, many neural and cognitive issues are diagnosed or monitored using written or verbal questionnaires and the subjective assessment or professionals examining a patient. Further, existing clinical tools such as physical exam, and central nervous system (CNS) imaging (computerized tomography (CT) scan or magnetic resonance imaging (MRI)) are subjective, not widely available, not sensitive or specific enough and too costly to identify all patients with CNS injury and therefore has a high false negative rate. This can include individuals on life support or cardiopulmonary bypass, trauma, loss of oxygen, etc. regardless of the initial injury or disease. Moreover, complex imaging modalities like CT scans and MRIs are far too expensive and of limited availability to provide a reasonable way for ongoing monitoring of an individual who otherwise shows none or limited signs of neural degradation.

### SUMMARY

The present invention includes systems for assessing a subject's cognitive status using a simple EEG recording. The presently disclose methods and systems use a novel arrangement of parallel pre-processing streams to remove different types of artifacts from neural data recorded by an EEG. Using the methods disclosed herein, the preprocessed data are used to provide a top-level assessment of a subject's cognitive status. Moreover, the presently disclosed systems and methods may use SHapley Additive exPlanations (SHAP) scores to parse model predictions and assess their confidence, which may help provide a deeper look at the underlying aspects of the top-level assessment.

In certain aspects, the present disclosure provides a system for assessing the cognitive status of a subject, the system comprising: a central processing unit (CPU); and storage coupled to said CPU for storing instructions that when executed by the CPU cause the CPU to: accept as an input, neural data from a subject, recorded using EEG sensors, said neural data; select EEG channels of interest from the neural data transform the neural data of each channel into a time series; input the time series into a first and second preprocessing stream, wherein: the first preprocessing stream: (i) performs a wavelet transformation of the time series to yield an amplitude time-course for each EEG channel and frequency of the neural data; (ii) calculates the dominant frequency and highest instantaneous power for each time-point of each time-course to identify momentary artifacts of the recorded neural data in the time series; and the second preprocessing stream: applies a bandpass filter and/or identifies and interpolates faulty channels; and remove the identified momentary artifacts from the time series input into the second preprocessing stream to produce preprocessed time series.

In certain aspects, the first preprocessing stream: (i) performs a wavelet transformation of the time series to yield an amplitude time-course for each EEG channel and frequency of the neural data; (ii) calculate a dominant frequency at each time-point of each time-course; (iii) determine the highest instantaneous power at each time-point; (iv) for each EEG channel, provides the dominant frequency and highest instantaneous power to an isolation forest model, thereby training a channel-specific model for each EEG channel that obtain an anomaly score for each time-point; (v) combine the anomaly scores for each time point to produce anomaly score time-courses; subject to the anomaly score time-courses to a threshold using cutoff values for each channel to produce binary vectors; and (vi) use the binary vectors for each channel to identify momentary artifacts in the time series.

In certain aspects, the wavelet transformation is a Morlet wavelet transformation. In certain aspects, the wavelets span the frequency bands of about 40 Hz to about 100 Hz. In certain aspects, the momentary artifacts comprise muscle artifacts in the recorded neural data.

In certain aspects, the CPU further: bandpass filters the preprocessed time series to produce a plurality of different frequency band time series; repeatedly advance a sliding-window along each frequency band time series and obtain at least one fractal measurement to produce at least one measure time series for each frequency band time series; extract measures from each measure time series; combine the extracted measures and analyze the combined measures using a machine learning system trained to correlate features in the combined measures with cognitive status to produce at least one Fractal Dimensions Distribution (FDD) score; and provide an output of the subject's cognitive status based on the FDD score.

In certain aspects, producing the FDD score comprises determining summary statistics for the distribution of the fractal measurements. In certain aspects, the summary statistics comprise one or more of a standard deviation of the fractal measurements, mean of the fractal measurements, skewness of the fractal measurements, and kurtosis of the fractal measurements. In certain aspects, the extracted measures comprise one or more of: frequency and/or time frequency measures; oscillatory measures; amplitude modulation measures; spectral connectivity measures; network analysis measures; chaos measures; complexity measures; and entropy measures.

In certain aspects, the output comprises a top-level, all cause assessment of the subject. In certain aspects, the output further comprises Shapely Additive explanation (SHAP) scores of a per-feature basis. In certain aspects, the SHAP scores are subject to semantic separation to provide semantic-feature grouping SHAP scores. In certain aspects, the semantic-feature grouping SHAP scores maintain directionality and magnitude consistent with the sum of the individual features and an overall model prediction probability. In certain aspects, the semantic feature-grouping SHAP scores are bounded with a 0 point marking a change in the directionality of the group influence on the top-level prediction.

In certain aspects, the system allocates a continuous score of the output into one of a plurality of bins, wherein each bin corresponds to a subject's risk of developing a cognitive impairment. In certain aspects, the neural data is collected from the subject over at least one period of time during which the subject is performing a task. In certain aspects, the recorded neural data includes event data correlated with performance of the at least one task. In certain aspects, the fractal measurement is a calculated a Katz fractal dimension (KFD). In certain aspects, the fractal measurement is a calculated Higuchi fractal dimension (HFD).

In certain methods, the neural data is recorded from the subject over at least one period of time during which the subject is at rest. In certain methods, the neural data is recorded from the subject over at least one period of time during which the subject is performing a task. In certain aspects, the recorded neural data includes event data correlated with performance of the at least one task. In some methods, the neural data is recorded from the subject during at least two periods of time. The methods may also include providing an output of the subject's cognitive status after every period of time during which neural data is recorded from the subject. Such methods include combining the results from each output to produce a longitudinal assessment of the subject's cognitive status.

Methods of the invention may further include annotating the recorded neural data with one or more annotations identifying one or more of the subject's age, sex, medical history, results from one or more biomolecular assay, and/or subjective cognitive assessment. The annotations may be provided to the machine learning system for analysis with the combined extracted measures.

### BRIEF DESCRIPTION OF DRAWINGS

FIG. 1 shows a workflow in accordance with the invention.

FIG. 2 shows the use of a sliding-window to produce a measure time series.

FIG. 3 shows an exemplary system of the invention.

FIG. 4 shows a map of EEG sensors.

FIG. 5 shows an exemplary Braiwell report.

FIGS. 6A-6B show sections from a Brainwell report on a mobile device.

### DETAILED DESCRIPTION

The present disclosure includes systems for assessing a subject's cognitive status using a simple EEG recording. The presently disclose methods and systems use a novel arrangement of parallel pre-processing streams to remove different types of artifacts from neural data recorded by an EEG. Using the methods disclosed herein, the preprocessed data are used to provide a top-level assessment of a subject's cognitive status. Moreover, the presently disclosed systems and methods may use SHapley Additive exPlanations (SHAP) scores to parse model predictions and assess their confidence, which may help provide a deeper look at the underlying aspects of the top-level assessment.

Methods disclosed herein use EEG recordings of neural data and may further include automated information gathering via self-report, self-assessment, questionnaires, cognitive and neuropsychological testing, and machine learning to render a report that provides insight into the cognitive and neural health of a user.

In certain aspects, to gather the necessary data for an assessment from a user, the methods and systems of the invention may include a CPU executing customized instructions and/or software custom software, an EEG cap, an EEG amplifier, and/or a testing computer (e.g., a laptop computer). To process the gathered data and render the report, the methods and systems of the disclosure may use a central set of servers (“cloud”) with custom software that processes EEG data and integrates it with non-EEG data such as user demographics (e.g., age and sex), self-assessment, questionnaires, and user performance during cognitive and neuropsychological testing. Custom machine learning software, which may be housed in the cloud servers, uses automated processing pipelines to process and transform the data, machine learning models produce output and the invention renders a report which can be accessed, e.g., via an internet browser or as a static file, such as a PDF.

The CPU of the testing computer (e.g., a laptop) may guide an operator in running a session for a user. The session may include capturing data a user inputs into software-based forms, preparing a user for EEG recording (e.g., operator fitting them with an EEG cap, applying a conductive gel when using a gel-based EEG), starting the recording, guiding the user through various tasks while EEG is being recorded, and completing the EEG recording and cleaning up.

Systems and methods of the disclosure may use an EEG cap connected to an amplifier, which is connected to the testing computer. The EEG cap detects changes in voltage amplitudes at the scalp and sends it to the EEG amplifier. The amplifier digitizes the signal and sends it to the testing computer where it is recorded as a file. The software injects event markers into the recording which denotes the beginnings and endings of tasks that the user was doing during recording which are recorded along with the EEG data. Tasks include “resting state”, in which the user has their eyes open and while they have their eyes closed while they are sitting or while they are standing. The invention can record zero or more blocks of each type of resting state tasks (eyes open/closed while sitting/standing). The tasks can also include cognitive and neuropsychological testing undertaken during EEG recording which can be presented on the computer recording the EEG signal or on a second computer.

The cognitive and neuropsychological tests help assess patient cognitive function in domains such as memory, executive function, learning, cognitive flexibility, intelligence, attention, and emotional wellbeing using tasks such as canonical versions or variants of Rey Auditory Verbal Learning Task, Wisconsin Card Sorting Task, Stroop, Simon Effect, Raven's Matrices, etc. In an exemplary embodiment, the EEG recording with event markers denoting what task the user was doing during a section of the EEG recording is then automatically transmitted to a central set of servers (“cloud”) for processing.

A CPU processor, such as one in a server of the cloud, may split the EEG recording up based on event markers and submits the segmented data to a preprocessing pipeline. The preprocessing pipeline is a collection of steps following data collection that help to clean the data to better enable feature generation.

Preprocessing can include a variety of different steps, and different preprocessing pipelines may be used to compute different features (i.e., by varying what preprocessing steps are included based on the feature that will be computed).

In one exemplary implementation, the first step is to select the relevant channels of interest. Then, notch filters may be applied to remove electrical noise from standard alternating current frequencies (exemplary implementation targets USA-typical line noise at 60 Hz and 120 Hz). A dual-stream processing pipeline is applied to remove multiple types of artifacts in parallel when these processes would conflict with each other if performed in series.

One stream of the dual-stream processor removes momentary artifacts that briefly contaminate the EEG data. In this exemplary implementation, there are two types of artifacts being identified and removed during this step. First the invention removes muscle artifacts, bursts of high-frequency activity which are typically seen due to fidgeting or scratching by the study participant (see below for details of how the system accomplishes this). Next the system removes segments of time in which the peak-to-trough amplitude within a brief sliding window (exemplary implementation; 500 ms) exceeds a preset threshold. These artifacts may reflect movements of the electrodes, or muscle movements, or amplifier glitches. The preset threshold is determined as a Z-score, and can optionally be tuned to find the threshold that optimizes performance of the report (exemplary implementation; Z=15). The dual-stream processor may also remove step-change artifacts, in which the voltage suddenly jumps from one sustained level to a different level.

In the second parallel stream, systems and methods of the disclosure may perform preprocessing such as a band-pass filter (e.g., from 0.1-50 Hz), or identification and interpolation of faulty channels.

At the end of the dual-stream processor, the time-points containing momentary artifacts from the first stream are removed from the EEG signals resulting from the second stream. Following the dual-stream processing, EEG signals may be re-referenced to the average across sensors, then converted into epochs. Each epoch is representative of a time-period of data collection where the participant was instructed to do a specific task such as sitting still with eyes open. Epoch boundaries are well defined by the events injected into the recording by the system's custom software during recording. Next, eye-movement activity is removed from the data using individual component analysis (ICA). In this stage, we find independent components separately for each epoch type (e.g., resting EEG with eyes open, resting EEG with eyes closed), and then automatically identify components that are strongly correlated with predetermined templates. These templates describe independent components that effectively capture EEG activity related to vertical and horizontal eye movements. Independent components matching these templates are then set to zero, and the data are re-projected from component space into sensor space. This allows the invention to separate out the influence of brain activity from eye movements, and to compute features independently for each signal. Finally, the peak-to-trough technique is applied again to identify large amplitude fluctuations within short windows of time. In different instances, this preprocessing pipeline may apply these steps in a different order, or may omit some steps, or apply them with different parameters.

The presently disclosed systems and methods may implement this novel, automated two-pipeline pre-processing to deal with muscle artifacts. Muscle artifacts represent broadband noise that contaminates signal and therefore causes error in the estimate of many types of features (e.g., spectral power, aperiodic signal, fractal dimension distributions, network connectivity, etc.).

Muscle artifact removal is an important step towards cleaning the EEG data. Methods and systems of the disclosure may apply the novel preprocessing technique described herein to identify segments of time contaminated by muscle artifacts, and remove those segments from the data. This technique relies on finding periods of time dominated by unusually high-frequency activity.

In certain methods and systems, following complete data acquisition, Morlet wavelets with a range of frequencies are convolved with each EEG channel. These wavelets span the frequency band that muscle artifacts (40-100 Hz) most strongly contaminate as well as lower frequencies that are influenced by muscle activity (10-30 Hz) to a lesser degree. For example, this system may use wavelets with frequencies ranging from 10 to 180 Hz in steps of 10 Hz, with wavelet lengths of 4 cycles for all frequencies. This wavelet transform yields an amplitude time-course for each channel and each frequency. The resulting amplitude time-courses are used to compute two signals that serve to identify muscle artifacts. First, is calculating the dominant frequency: the frequency with the highest power at each time-point. Second, at each time-point, determining the power of the amplitude time-course above a predetermined frequency threshold (e.g. 70 Hz) that has the highest instantaneous power. These two signals are computed for each timepoint for each channel used in the EEG dataset. These two signals are then provided to an isolation forest model, which is commonly used for outlier detection.

A separate isolation forest model is trained for each EEG channel, using the feature vector of dominant frequency and peak high-frequency power at each time-point. Next, the channel-specific models are used to obtain the isolation forest anomaly score for each time-point. The, systems and methods of the disclosure take the maximum anomaly scores across a rolling time-window (e.g. 250 milliseconds) which helps to remove the tapered-off beginnings and ends of muscle artifacts. Next, these anomaly score time-courses are thresholded using predetermined cutoff values for each channel.

The result of these cutoffs is a binary vector—predicting either clean or artifact-contaminated data for each time-point. Finally, the individual channel predictions are combined. If the number of channels at which an artifact is detected in a given time-point is above some predetermined threshold (e.g., 3 channels), then that time-point is deemed as artifactual and the time-series values are set to NaN (Not a Number), excluding them from subsequent computations. Employing the novel muscle artifact pipeline improves estimates of feature values from EEG data and improves the overall accuracy of the system's machine-learning predictions.

Systems and methods of the disclosure may establish muscle artifact cutpoints and thresholds prior to application, e.g., use to assess the cognitive status of an individual.

In certain aspects, this include the isolation forest anomaly score thresholds being determined via a semi-automated procedure that maximizes muscle-artifact detection accuracy in a manually-tagged example dataset. This example dataset is derived using the same EEG recording equipment as the main data to be classified. Based on agreement with ground truth-annotations and algorithm predictions, the performance was optimized using the F1-score (TP/(TP+0.5*(FP+FN)). Optimized cutoff values for each channel were stored and used as templates for future data collection utilizing the same data-collection devices.

During the preprocessing approach described above, momentary artifacts in the EEG data are replaced with NaNs (markers indicating that the value at a particular time-point is “not a number”). These NaNs help to ensure that artifacts do not contaminate our EEG metrics, but they also create challenges for EEG processing. Some computations fail entirely when a signal contains any NaNs, while other computations propagate those NaNs across the whole time-series (causing all data to be lost). The methods and systems of the disclosure provide a system that may replace artifact-contaminated time segments with NaNs, while still performing the data processing steps that would be impossible with data that contain NaNs. This system is referred to herein as the “dual-stream processor”. In this system, processing steps that create NaNs in the signal (NaN-creation), such as muscle artifact identification, are applied in parallel to processes that require no-nans to be present (NaN-avoidance). The original EEG data is ingested and sent into two processing pipelines, simultaneously. After the two processing streams are completed, the data from the NaN-creation process is converted into a boolean mask. The boolean mask is then applied to the output of the NaN-avoidance process to create a single data output that incorporates both NaN-creating and NaN-avoiding processing.

In certain aspects, the presently described systems and methods may use machine learning models to consider classes of features such as speed and accuracy during cognitive and neuropsychological testing, self-report data provided by the user, demographics, medical history. The systems and methods may also incorporate a multitude of features computed from EEG data, which can include spectral power, aperiodic activity, measures of complexity, entropy, network behavior, graph representations, microstates, and descriptive statistics of raw signal. The systems may compute the same metric from EEG recorded under different task conditions; e.g., alpha power computed from resting state EEG recorded with the user sitting with their eyes open, and alpha power computed while the user was performing a cognitive testing task.

In certain aspects, the presently described systems and methods includes systems and methods for assessing the cognitive function in a subject use the novel Fractional Dimensional Distributions (FDD) technique. FDD provides a new class of measures that have proven effective in detecting the presence of neurodegeneration in subjects using only non-invasive brain imaging techniques. The present Inventors developed the FDD-based methods and systems of the invention on the insight that brains of those experiencing cognitive impairments may have distinct troubles sustaining complex activity, and that activity in different oscillatory bands is differentially impacted by the progressions of different cognitive impairments. The presently disclosed FDD techniques measure stability of the complexity of a brain's activity within particular oscillatory bands. It therefore makes a surprising improvement on both approaches that summarize brain activity complexity without regard to the moment-to-moment changes in a brain's ability to sustain that complexity, as well as naïve approaches that consider only the spectral power of neural activity in different oscillatory bands without regard to the complexity of that oscillatory activity.

Neurotypical brains sustain complex activity that shows similarity across different time scales, which is analogous to the way fractals display similarity at different spatial scales. Signal processing measures such as Katz Fractal Dimension and Higuchi Fractal Dimension have been developed to characterize that type of activity within time-varying signals. This family of signal processing techniques have been applied to neuroimaging recordings to characterize the complexity of brain activity. When applied to EEG recordings, the resulting fractal measure values have been shown to be helpful in classifying healthy patients versus those with cognitive issues.

A general approach is to compute the fractal measure on an entire EEG recording at each sensor, yielding 1 value per EEG sensor per subject (e.g., 19 HFD values computed from a 5-minute EEG recording using a 19-channel cap), a whole-recording wideband technique.

In certain aspects, the presently disclosed systems and methods using FDD separate a time series of neural activity (e.g., from an EEG recording) into frequency-banded time series, to measure stability of the complexity of a brain's activity within different, particular oscillatory bands. Thus, the systems and methods of the invention improve upon prior approaches that summarize brain activity complexity without considering to the moment-to-moment changes in a brain's ability to sustain that complexity, or naïve approaches that consider only the spectral power of neural activity in different oscillatory bands without considering to the complexity of that oscillatory activity.

Although the methods and systems using FDD described herein are preferably used with neural data provided by EEG recordings, the FDD may also be computed using timeseries of neural activity such as that derived from MEG, fNIRS, or MRI.

The present Inventors have discovered that, surprisingly, by using the systems and methods of the disclosure, the cognitive status of a subject may be determined with an accuracy of 96%, sensitivity of 90%, and specificity of 99%.

In certain aspects, the systems and methods of the invention employ a classifier that includes one or more machine learning models.

Machine learning (ML) is branch of computer science in which machine-based approaches are used to make predictions. (Bera et al., Nat Rev Clin Oncol., 16(11):703-715 (2019)). ML-based approaches generally require training a system, such as a familiarity classifier, by providing it with annotated training data. The system learns from data fed into it, to make and/or refine predictions. Id. Machine learning differs from, rule-based or statistics-based program models. (Rajkomar et al., N Engl J Med, 380:1347-58 (2019)). Rule-based program models rely on explicit rules, relationships, and correlations. Id.

In contrast, an ML model learns from examples fed into it, and creates new models and routines based on acquired information. Id. Thus, an ML model may create new correlations, relationships, routines or processes never contemplated by a human. A subset of ML is deep learning (DL). (Bera et al. (2019)). DL uses artificial neural networks. A DL network may include layers of artificial neural networks. Id. These layers may include an input layer, an output layer, and multiple hidden layers. Id. DL is able to learn and form relationships that exceed the capabilities of humans. (Rajkomar et al. (2019)).

By combining the ability of ML, including DL, to develop novel routines, correlations, relationships and processes amongst complex data sets, such as the EEG recordings used in the systems and methods of the inventions, the classifiers described herein can provide accurate and insightful assessments neural data to provide an accurate assessment of a subject's cognitive status.

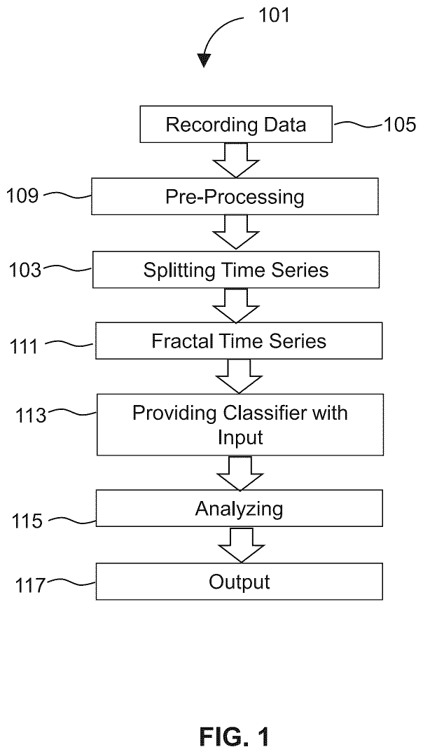

FIG. 1 provides a generalized workflow 101 used in connection with the systems and methods of the invention. As shown, the workflow 101 requires recording neural data from a subject 105, preferably using EEG electrodes placed on the subject's scalp.

In certain methods and systems, the EEG measurements obtained from the subject undergo a pre-processing step 109, such as the dual-stream preprocessing step described above. Methods and systems of the invention may employ a number of pre-processing techniques, including but not limited to, amplifying EEG recorded voltages, converting analogue EEG signals into digital signals, filtered, bandpass filtered, baseline corrected, referenced, referenced, normalized. This pre-processing step 109 converts the raw electrical voltage potentials from the EEG neural recording into a time series. Preprocessing may include a variety of steps, analytical components, hardware modules and/or software operations.

In preferred systems and methods, preprocessing 109 includes removing artifacts, such as electromyographic artifacts those caused by a subject's behaviors (e.g., muscle contractions and blinking or moving their eyes), environmental artifacts (e.g., a door closing or leaky electronics use nearby during recording), and other artifacts or “jumps” produced during use of the high-impedance electrodes of an EEG.

In preferred systems and methods of the invention, EEG data is preprocessed using an automated dual-stream pipeline. The pipeline may interpolate any brief jump artifacts present, which may be identified.

In certain aspects, the data may be band-pass filtered. In certain aspects, the data is re-referenced to the common average. Methods and systems may use template-matching to identify artifacts in the raw EEG data. Aspects of the EEG data may be compared with templates characteristic of one or more EEG artifacts. If the compared aspects of the raw EEG match such a template, an artifact may be identified in the raw EEG data. Certain methods and systems use Independent Component Analysis (ICA) in which components from the raw EEG data are extracted and compared to the same components in EEG artifact templates. In certain aspects, the ICA extracts at least 1, 2, 3, 4, 5, 6, 7, 8, 9, 10, 11, 12, 13, 14, 15, 16, 17, 18, 19, or 20 components from the raw EEG data. Certain methods and systems may use an automated Independent Component Analysis (ICA) method that uses template-matching to identify and remove ICA components that captured EEG artifactual activity created, for example, by a subject's eye saccades and eye blinks. Templates, e.g., indicative of eye movement or inter-system artifacts, may include a number of components identifiable by ICA extraction and analysis.

In certain aspects, artifacts may be removed and replaced with a linear interpolation between the nearest samples that were not contaminated by artifacts. In preferred aspects, the linear interpolation is computed separately for each channel.

The time series data can further be further transformed during the pre-processing step, for example by a Fourier transform or fast Fourier transform, into a spectral and/or time-frequency domain.

The pre-processing step 109 may further include normalizing neural data at one or more specific frequency to that of another frequency. For example, in preferred methods and systems, neural data at 6 Hz is used to normalize the neural data in the frequency band time series.

Returning to the method described in FIG. 1, after pre-processing 109 and splitting/filtering 103 the whole-recoding time series into frequency band time series. The systems and methods of the invention using FDD produce 111 at least one fractal time series from each frequency band time series.

In certain aspects, producing the at least one fractal time series 111 includes assigning windows or bins to each frequency band time series. Typically, window or bin applies to a subset of a set of data with the implication that, for linear (e.g., over time) data, values of those data will be put into sets, dubbed windows or bins (that may or may not overlap), where those sets are suitable to an analysis as inputs for the classifiers disclosed herein.

Preferably, the window size is tunable. An exemplary system of the invention may use a window with 1 second of data (500 samples). After computing the measures within a given window, the system advances the window. The amount of time or samples the window is advanced is tunable and is 100 ms (50 samples) in an exemplary system. The system then computes the measures again in the new window. This process continues until the entire timeseries has been processed through the sliding window approach, yielding measure/fractal timeseries.

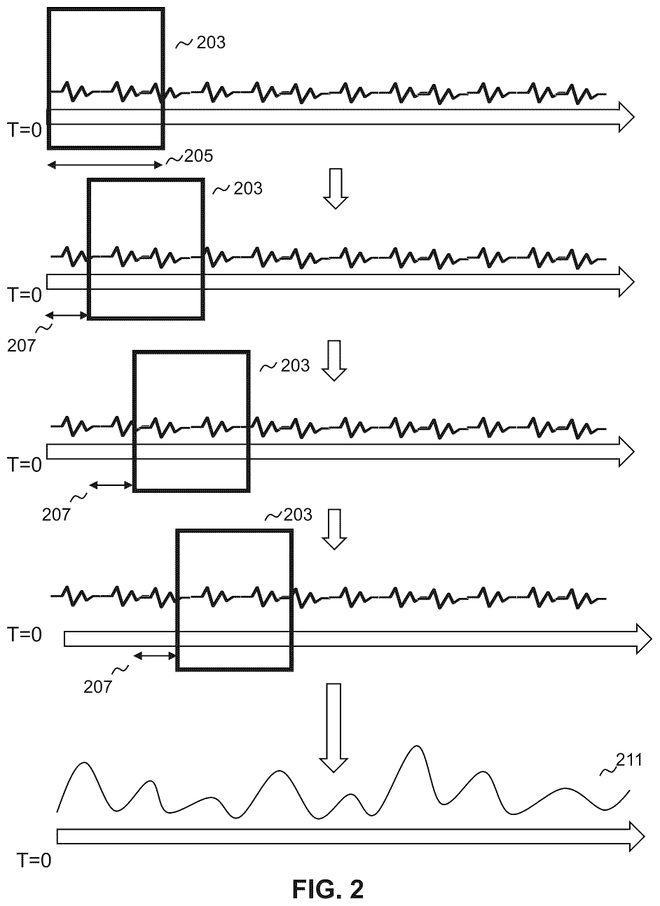

FIG. 2 provides a schematic for producing a fractal time series. In FIG. 2, a sliding-window 203 is applied to a frequency band time series. The sliding-window includes a size 205 in time. For example, in preferred aspects, a window has a size of around 1 to 100 second. In certain aspects, the window has a size of around 1-2 seconds. The window 203 also has a step/stride 207. As shown in FIG. 2, the step/stride 207 is the amount of time along the time series that the window is advanced.

In preferred aspects, at each step/stride at least one fractal measure is obtained from the time series. In certain aspects, a plurality of fractal measures is obtained from each time series at each step/stride. The value of the fractal measure(s) obtained at each step is recorded as the window slides across the frequency band time series. The saved values are a new timeseries of fractal values that captures the stability of the complexity of the brain's activity within a particular oscillatory band. As a result, the fractal measures for each frequency band time series are used to create a measure/fractal time series 211 from each frequency band time series.

In an exemplary method the recording is filtered into different frequency bands (e.g., delta, theta, alpha, beta1, beta2, gamma), yielding as many timeseries as band filters. Within each filtered timeseries the data is transformed into a timeseries of fractal values using the sliding window technique described in FIG. 2. The window is defined by the amount of neuroimaging timeseries data it captures (e.g., two seconds of timeseries) and a step size that defines the amount of time the window is advanced along the timeseries at each step (e.g., 50 ms).

The window initially captures the first two seconds of a timeseries. A fractal measure such as the Higuchi fractal dimension (HFD) is computed from the data within the window, and the resulting HFD value is saved. Then the window is advanced by the step size along the filtered neuroimaging timeseries, and a new HFD value is computed and saved. This is repeated along the filtered timeseries until the final sample has been included once in a fractal computation. The saved values are a new timeseries of fractal values that captures the stability of the complexity of the brain's activity within a particular oscillatory band.

In certain aspects, in each window slid along a frequency band time series, more than one fractal measure is obtained at each step. In certain aspects, different fractal measures are used to produce different fractal/measure time series from each frequency band time series. In certain aspects, a combination of fractal measures are combined to produce a fractal/measure time series from each frequency band time series.

Exemplary fractal measures used in the methods and systems of the invention include one or more complexity measures such as the Higuchi fractal dimension (HFD) and the Katz fractal dimension (KFD). The HFD is nonlinear measure for how much variation there is in the signal. When the signal is rhythmic with repeating patterns, HFD is low. However, if the signal is more complex with more variations and less repetition, HFD is high. Similar to HFD, KFD also measures the self-similarity of a signal. However, HFD does so by subsampling the signal and analyzing the signal similarity within each subsample. KFD involves calculating the average distance between successive points in a signal.

In certain aspects, the after dual-stream pre-processing the fractal measures are obtained as described by the present Inventors in PCT/US2023/12274, which is incorporated by reference herein in it entirety.

In certain aspects, the methods and systems of the invention obtain one or more of Lyapunov Exponent (LE), Hjorth mobility (HM), Hjorth complexity (HC), sample entropy (SaE), spectral entropy (SpE), approximate entropy, multiscale entropy (MSE), permutation entropy (PE) and Hurst exponent (HE) from each window to produce a fractal/measure time series.

Lyapunov Exponents measure growth rates of generic perturbations of a dynamical system.

Entropy is a concept in information theory that quantifies how much information is in a probabilistic event. The more predictable an event is (extremely high or extremely low probabilities), the less information there is and therefore the lower the entropy values. SpE, also known as the Shannon Entropy of the spectrum that measures how flat the spectrum is: the higher the value, the flatter the power spectrum (which means there are less peaks in different frequency components). Approximate entropy is a measure to quantify the amount of regularity and predictability in a signal, similar to a complexity measure.

SaE is an improvement of approximate entropy with decreased bias and more accurate estimate of the complexity of a signal. Similar to HFD, it also subsamples the signal and calculates distance metrics for the subsamples. Multiscale entropy is an extension of sample entropy (or approximate entropy) where instead of just calculating sample entropy based on single samples in the EEG data, it also time windows with varying window lengths and calculates the sample entropy of those time windows. MSE potentially contains more information than just the sample entropy: it also measures self-similarity or complexity of the signals for longer time ranges.

Permutation Entropy subsamples a signal, orders the subsample by magnitude and summarizes the ordering information within the entire signal, which quantifies the pattern of change in the signal.

In certain aspects, the system performs a frequency decomposition via a Hanning-windowed Fourier transform on the fractal/measure timeseries to characterize oscillations in how a given measure at a given location changed over the recording period. For instance, the system can compute the strength of delta oscillations in the beta-band filtered Katz Fractal Dimension time series that was computed from a timeseries source localized to the left hippocampus.

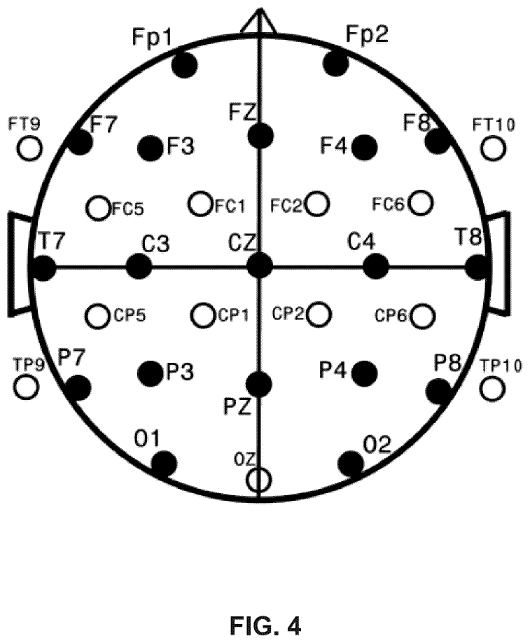

FIG. 4 provides a schematic of a 10-10 system multi-channel EEG array positioned on the head of a subject. Although this schematic shows a 10-10 multi-channel EEG, other EEG systems, such as 10-20 and 10-5 systems, can be used with the systems and methods of the invention.

As shown in FIG. 4, the 10-10 system includes 32 electrodes that are positioned on the scalp of a subject. Each electrode is identified according to the standard 10-10 system nomenclature. In certain aspects, when using a 32 electrode array neural data recorded from a subset of sensors includes data recorded from a subset that includes 1 electrode, or 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 electrodes of the array.

In certain aspects, the pre-processing step 109 includes referencing the recorded neural data by one or more EEG sensor to that recorded by one or more other sensors of the array. In certain aspects, the neural data recorded by a sensor or subset of sensors is referenced to a single other sensor of the array. In certain aspects, the single other sensor of the array is a vertex sensor, such as CZ on a 10-10 array. For example, in certain aspects, neural data recorded from a subset of about 8 bilateral and sagittal midline sensors is referenced to a vertex sensor, such as the C_{Z} on a 10-10 array. After referencing, the data recorded from the referenced sensor(s) may be discarded and not used as an input for the familiarity classifier.

In certain aspects, the pre-processing step 109 includes annotating data provided as an input to a classifier used in the systems and methods of the invention, including when used as training data. Annotation can be performed automatically by the systems of the disclosure and/or by human action or direction. Annotations can be used as features by the classifier to discern or create correlations regarding cognitive status and recorded neural data using the FDD technique.

Annotations may additionally or alternatively include, for example, the date, time, or location of a cognitive assessment or actions performed during an assessment. Annotations may include information derived or obtained from Electronic Medical Records (EMR) or clinical trial records. In certain aspects, annotations may include the results of a subjective cognition test and/or determined cognitive impairment.

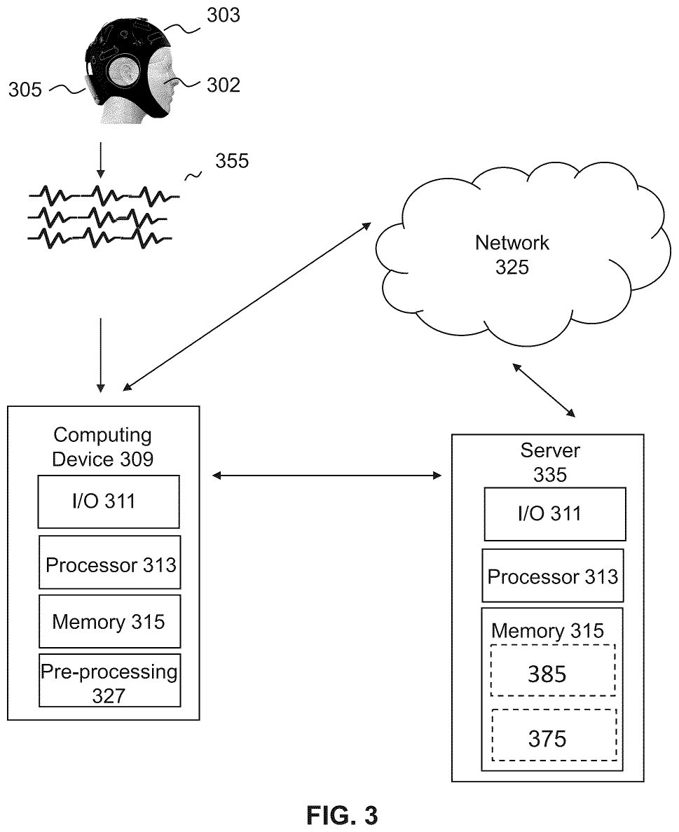

FIG. 3 shows select components of an exemplary system of the invention. The system an array 303 of EEG sensors that are positioned on the subject's scalp, and a computing device 309.

Preferably, the array 303 of EEG sensors are incorporated into a cap such as the 21, 25, 32, and 64 channel Waveguard™ EEG caps (ANT Neuro, Hengelo, Netherlands). Generally, EEG sensors are placed on bony structures on a subject's scalp. By using an EEG cap, the sensors are correctly positioned on the subject's scalp, eliminating the need to spend time carefully positioning the sensors.

The array/cap 303 may include an amplifier 305, which may include one or more filters. The amplifier 305 amplifies the raw potentials recorded from the sensors of the array/cap 303. The amplifier 305 may be connected to, or include, a programmable analogue/digital converter.

The array/cap 303 interacts (e.g., exchange data with) the computing device 309. The array/cap 303 may be connected to the computing device 309 via a wired connection. Alternatively, the array/cap 303 and computing device 309 exchange information using any combination of a local network (e.g., “Wi-Fi”), the internet, satellite, cellular data, or a short-range wireless technology, e.g., Bluetooth®. Neural data 355 from the subject is recorded using the array/cap 303 and transmitted to the computing device 309.

The computing device 309 may function as a remote or networked terminal that is in connection with a centralized computer system. Thus, the system may include one or more server computers 335 in communication with the computing device 309, preferably via connection with a network 325.

Each computing device 309 and server 325 includes a processor 313 coupled to a tangible, non-transitory memory 315 device and at least one input/output device 311. Thus, the system includes at least one processor 325 coupled to a memory subsystem 315. The components may be in communication over a network 325 that may be wired or wireless and wherein the components may be remotely located or located in close proximity to each other.

As shown in FIG. 3, the memory subsystem 315 of the server 309 includes one or more classifier 375 comprising one or more machine learning models. In certain aspects, the classifier 375 is hosted on a server 309 in communication with the computing device 309.

Processor refers to any device or system of devices that performs processing operations. A processor will generally include a chip, such as a single core or multi-core chip (e.g., 12 cores), to provide a central processing unit (CPU). In certain embodiments, a processor may be a graphics processing unit (GPU) such as a NVidia Tesla K80 graphics card from NVIDIA Corporation (Santa Clara, CA). A processor may be provided by a chip from Intel or AMD. A processor may be any suitable processor such as the microprocessor sold under the trademark XEON E5-2620 v3 by Intel (Santa Clara, CA) or the microprocessor sold under the trademark OPTERON 6200 by AMD (Sunnyvale, CA). Computer systems of the invention may include multiple processors including CPUs and or GPUs that may perform different steps of methods of the invention.

The memory subsystem 315 may include one or any combination of memory devices. A memory device is a device that stores data or instructions in a machine-readable format. Memory may include one or more sets of instructions (e.g., software) which, when executed by one or more of the processors of the disclosed computers can accomplish some or all of the methods or functions described herein. Preferably, each computer includes a non-transitory memory device such as a solid-state drive, flash drive, disk drive, hard drive, subscriber identity module (SIM) card, secure digital card (SD card), micro-SD card, or solid-state drive (SSD), optical and magnetic media, others, or a combination thereof.

The computing device 309 and server computer 335 may include an input/output device 311, which is a mechanism or system for transferring data into or out of a computer. Exemplary input/output devices include a video display unit (e.g., a liquid crystal display (LCD) or a cathode ray tube (CRT)), a printer, an alphanumeric input device (e.g., a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem.

Using one or more I/O device or connection, the computing device 309 can transmit instructions to the EEG array 303. Recorded neural data can be stored in the memory subsystem of the computing device 309. In certain aspects, the neural data is transmitted to a remote computing device 309 from a server computer 335 via a network 325.

In certain aspects, the system further includes one or more means, in addition to the EEG array 303, to provide biometric data from a subject undergoing a cognitive assessment. For example, the system may include an imaging subsystem 331, such as a camera or specialized eye tracking device. The imaging subsystem 331 may be used, for example, to track a subject's eye movement, or track micro-facial expressions (facial coding). Eye tracking can be used to provide additional data features analyzed by the familiarity classifier.

Surprisingly, the present Inventors have discovered that a specialized eye tracking device is not necessarily required for purposes of eye tracking. Rather, a digital camera, such as that integrated into a laptop serving as the computing device 309, can provide the necessary resolution for eye-tracking purposes.

Facial coding can be used, for example, to provide additional data regarding a subject's emotional response during a cognitive assessment. This data can be used by the classifier to increase accuracy and specificity.

As shown in FIG. 3, one or more of the computing device 309 or server computer 335 includes a pre-processing module 327. The pre-processing module 327 performs one or more of the pre-processing steps as described herein. Pre-processing modules may include a combination of hardware (e.g., filters and amplifiers) and software components. In certain aspects, the computing device 309 includes a pre-processing module 327, which allows the computing device to transmit pre-processed neural data to a server computer 335 for analysis by a classifier hosted thereon. By performing at least part of the pre-processing on the computing device 309, less network bandwidth is required for transmitting the neural data 355 to the server computer 335. Neural data digitized and/or filtered from its raw, analogue state, is far less data intensive.

In certain aspects, the computing system 309 includes one or more applications for initializing and/or calibrating aspects of the classifier for a cognitive assessment. For example, the computing system 309 may include instructions displayed to a subject undergoing an assessment regarding, for example, to close their eyes and remain still and how to affix the EEG cap 303. The instructions may task the subject with focusing on a certain spot on a screen, taking a physical action (e.g., standing or closing eyes), and the like. During this time, the computing system may, for example, calibrate the EEG array, an imaging subsystem (e.g., an eye tracking device) and/or obtain baseline neural data from the subject.

In certain aspects, neural data recorded during this time can be used to train a classifier. This training data can be used to train a classifier for use with other subjects. Alternatively or additionally, this training data is used to train the classifier that will analyze the subject's neural data for a cognitive assessment.

In certain methods and systems, the classifier 355 includes one or more machine learning (ML) model.

Systems and methods of the invention may produce an output that provides an assessment of a subject's cognitive after a neurological assessment using the techniques described herein.

In certain aspects, a trained model ingests the computed features and renders a “top level” output. The top level output provides an assessment of the cognitive and neural health of the person the EEG was taken from. Depending on the trained model that rendered the output, it may be binary or continuous. The output is an assessment of whether the user's data indicates cause for concern. The output represents an “all cause” assessment, and the output can be affected by, and therefore reflect sleep apnea, TBI, neurodegeneration, insomnia, depression, anxiety, schizophrenia, poorly controlled diabetes, stroke, vascular damage, consequences of seizure and other syndromes or diseases that affect the brain and cognition.

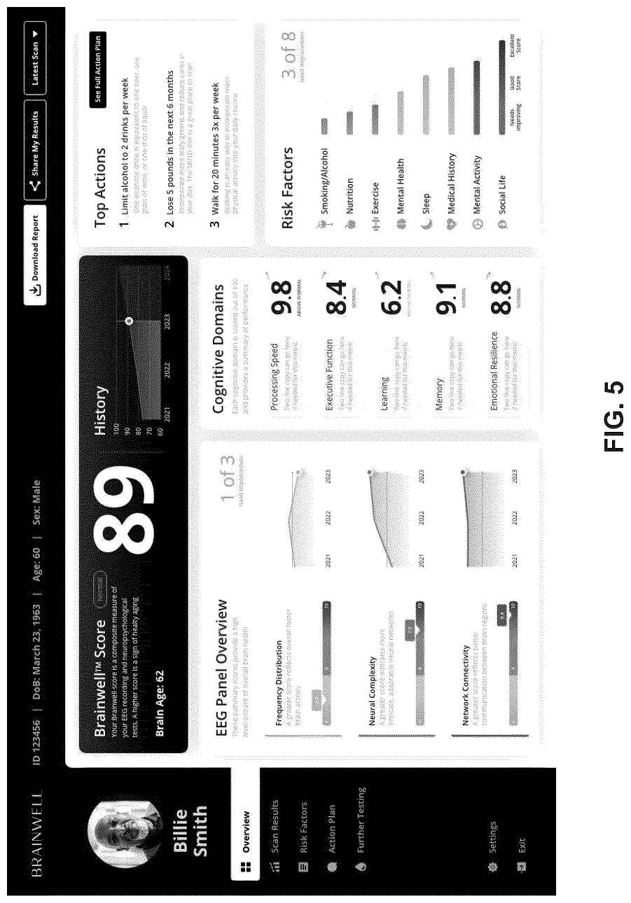

An exemplary report is shown in FIG. 5, hereinafter a “Brainwell report”, and contains several “tiers” of output that respectively provide insights that are broad and general as well as insight that are specific and narrow. The “top level” output described above is the core material for the most general insight in the report. The report also contains more detailed tiers of output that provide more specific insights. Ways systems and methods derive such more narrow and specific insights are described below.

In certain aspects, systems and methods of the disclosure use SHapley Additive exPlanations (SHAP) scores to parse model predictions and assess their confidence.SHAP scores are computed on a per-feature basis. This may make use of both the magnitude and direction of the score for understanding a feature's contribution to the prediction. A higher magnitude implies a greater influence on the prediction, while the sign of the SHAP score indicates the directionality of that influence on the prediction. The Brainwell report can then combine this information with scientific knowledge about the relationship between the feature or feature family with brain and cognitive health.

Given the large number of features used in EEG-models, it is difficult to explain the differences between individual features that are related to a similar type of neural characteristic, such as connectivity. Therefore, separating the features semantically into high-level groupings enables more digestible explainability. From the input features (where there are N features), semantic separation results into a few distinct groupings (i.e. M groupings where M<<N). The features within each grouping are summed together to create a semantic-feature-grouping SHAP score. These SHAP score groupings maintain directionality and magnitude consistent with the sum of the individual features and overall model prediction probability. The group SHAP score is then normalized to fall within a bounded range (e.g. −5 to +5) where the 0 point marks a change in the directionality of the group influence on the top-level prediction. To scale the grouping SHAP score into the modified grouping SHAP score, a universal edge per grouping is calculated, then multiplied by the arbitrary value X to ensure it falls within +/−X. The universal edge is defined as the absolute maximum value of the grouping SHAP score for any subject's data within the training dataset. For unseen data, the SHAP scores can be calculated and grouped using the pre-defined semantic-groupings and scaled using the universal edge, as determined by the training data. By combining the single feature SHAP-scores into modified semantic feature grouping SHAP scores, the systems and methods of the disclosure derive otherwise-unavailable insights from the model about specific neural characteristics of the user, and do so in a way that can be understood by users and laypeople generally.

As shown in FIG. 5, the Brainwell Report (“report”) provides an assessment of user cognitive and neural health. The report is rich and contains several “levels” or “tiers” of output. The top level provides a summary which is expressed either as “Cognitive Age” or a “Brainwell Score”. The Cognitive Age of a user will be lower, the same as, or higher than their actual age. The Brainwell Score is an alternative summary expression of user cognitive and neural health. The score is continuous and bounded (in the exemplary case, it falls between 1 and 100). A secondary level of the report breaks the summary down into feature-cluster summaries of user neural activity and assesses whether the cluster was indicating better or worse cognitive and neural health. Secondary level feature clusters can include things like spectral power, neural complexity, or network connectivity/inter-area brain coordination. A tertiary level of feature reporting breaks the secondary feature clusters into component features and assesses whether they indicated better or worse cognitive and neural health. For example, the tertiary level can include a report on the peak alpha power, which is a component of the secondary cluster of spectral power. The process of deriving these different tiers of insight (from the most general to more narrow and specific) is described above.

The report contains two additional related sections. One section provides information on user risk factors and the other section presents an action plan for the user. The risk factors and lifestyle data evaluated include but are not limited to: smoking, alcohol consumption, level of physical activity, nutritional intake, mental health status, and sleep patterns. The invention gathers this information during a recording session. In an exemplary implementation, the risk factor and lifestyle data is gathered before the EEG recording commences using the invention's custom software and uploads it to the cloud for processing. The report incorporates machine-learning output to identify potential risks to cognitive health and grade them in terms of severity and immediacy. The related action plan is personalized for each user. The action plan is tailored to their specific needs using data about the user's habits, risk factors, EEG data, and cognitive/neuropsychological testing results. The action plan includes targeted recommendations for lifestyle changes, exercises, diet, and more, with the aim of reducing or eliminating the impact of risk factors and promoting overall cognitive and neural health.

If the systems and methods of the disclosure are applied to a user more than once, reports generated for subsequent sessions will contain additional information reflecting the history of report contents. With respect to the risk factors and lifestyle data and related action plan, tracks user's progress and adjusts action plans over time to increase effectiveness and adaptability to the changing needs of the user. Subsequent reports contain an additional report section: the “change report”. The change report highlights important changes from the previous report(s) by considering the directionality and magnitude of feature scores as described above (as groups or single features, considered within the model's prediction context and rescaled). The report provides supplementary information about features and feature groups which can include explanatory prose and citations.

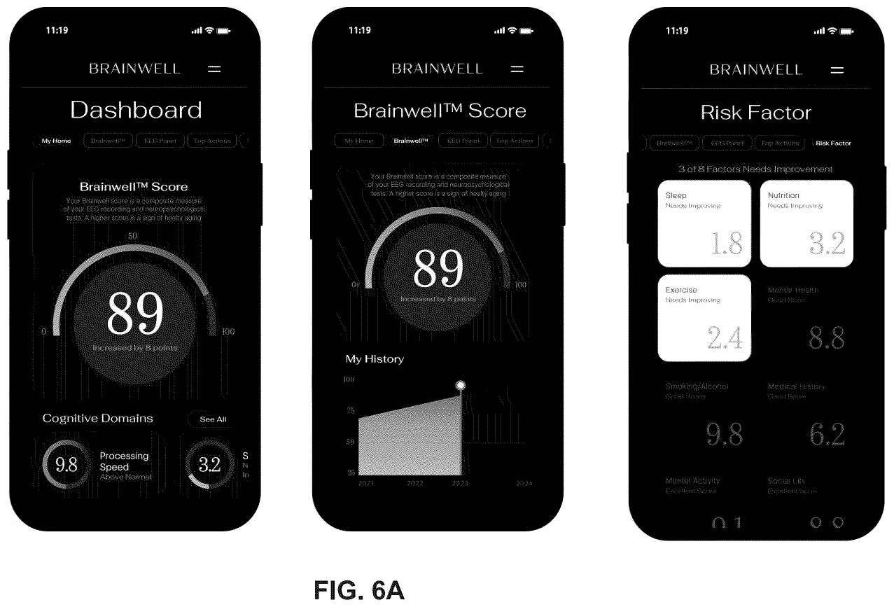

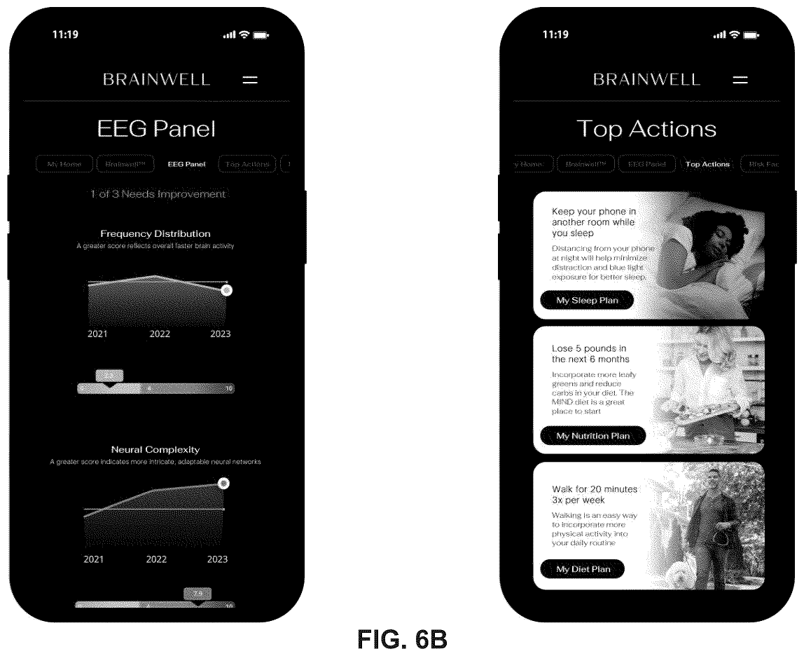

As shown in FIGS. 6A-6B, in certain aspects the aforementioned reports, or portions thereof, the action plan(s), change report, and the like may be provided to individual users remotely, e.g., via smart phone application.

Any of several suitable types of machine learning may be incorporated into one or more steps of the disclosed methods and systems. Classifiers of the invention may use machine learning approaches, which include neural networks, decision tree learning such as random forests, support vector machines (SVMs), association rule learning, inductive logic programming, regression analysis, clustering, Bayesian networks, reinforcement learning, metric learning, and genetic algorithms. One or more of the machine learning approaches (aka type or model) may be used to complete any or all of the method steps described herein.

For example, one model, such as a neural network, may be used to complete the training steps of autonomously identifying features in one or more subject's neural data and associating those features with the cognitive status of a subject (e.g., during training or calibrating). Once those features are learned, they may be applied to test samples by the same or different models or classifiers (e.g., a random forest, SVM, regression) for the correlating steps during a cognitive health assessment.

In certain aspects, features may be identified and associated with the cognitive status in a subject using one or more machine learning systems, and the associations may then be refined using a different machine learning system. Accordingly, some of the training steps may be unsupervised using unlabeled data while subsequent training steps (e.g., association refinement) may use supervised training techniques such as regression analysis using the features autonomously identified by the first machine learning system.

In decision tree learning, a model is built that predicts that value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, Random Forests, Machine Learning 45:5-32, incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable. Random forests can also be used to determine dissimilarity measurements between unlabeled data by constructing a random forest predictor that distinguishes the observed data from synthetic data. Id.; Shi, T., Horvath, S. (2006), Unsupervised Learning with Random Forest Predictors, Journal of Computational and Graphical Statistics, 15(1):118-138, incorporated herein by reference. Random forests can accordingly by used for unsupervised machine learning methods of the invention.

SVMs are useful for both classification and regression. When used for classification of new data into one of two categories, such as having a disease or not having the disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. SVMs can also be used in support vector clustering to perform unsupervised machine learning suitable for some of the methods discussed herein. See Ben-Hur, A., et al., (2001), Support Vector Clustering, Journal of Machine Learning Research, 2:125-137.

Regression analysis is a statistical process for estimating the relationships among variables such as features and outcomes. It includes techniques for modeling and analyzing relationships between multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

Association rule learning is a method for discovering interesting relations between variables in large databases. See Agrawal, 1993, Mining association rules between sets of items in large databases, Proc 1993 ACM SIGMOD Int Conf Man Data p. 207, incorporated by reference. Algorithms for performing association rule learning include Apriori, Eclat, FP-growth, and AprioriDP. FIN, PrePost, and PPV, which are described in detail in Agrawal, 1994, Fast algorithms for mining association rules in large databases, in Bocca et al., Eds., Proceedings of the 20th International Conference on Very Large Data Bases (VLDB), Santiago, Chile, September 1994, pages 487-499; Zaki, 2000, Scalable algorithms for association mining, IEEE Trans Knowl Data Eng 12(3):372-390; Han, 2000, Mining Frequent Patterns Without Candidate Generation, Proc 2000 ACM SIGMOD Int Conf Management of Data; Bhalodiya, 2013, An Efficient way to find frequent pattern with dynamic programming approach, NIRMA Univ Intl Conf Eng, 28-30 Nov. 2013; Deng, 2014, Fast mining frequent itemsets using Nodesets, Exp Sys Appl 41(10):4505-4512; Deng, 2012, A New Algorithm for Fast Mining Frequent Itemsets Using N-Lists, Science China Inf Sci 55(9): 2008-2030; and Deng, 2010, A New Fast Vertical Method for Mining Frequent Patterns, Int J Comp Intel Sys 3(6):333-344, the contents of each of which are incorporated by reference. Inductive logic programming relies on logic programming to develop a hypothesis based on positive examples, negative examples, and background knowledge. See Luc De Raedt. A Perspective on Inductive Logic Programming. The Workshop on Current and Future Trends in Logic Programming, Shakertown, to appear in Springer LNCS, 1999; Muggleton, 1993, Inductive logic programming: theory and methods, J Logic Prog 19-20:629-679, incorporated herein by reference.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node's parent variables, and gives (as output) the probability (or probability distribution, if applicable) of the variable represented by the node. See Charniak, 1991, Bayesian Networks without Tears, AI Magazine, p. 50, incorporated by reference.

A neural network, which is modeled on the human brain, allows for processing of information and machine learning. A neural network includes nodes that mimic the function of individual neurons, and the nodes are organized into layers. The neural network includes an input layer, an output layer, and one or more hidden layers that define connections from the input layer to the output layer. The neural network may, for example, have multiple nodes in the output layer and may have any number of hidden layers. The total number of layers in a neural network depends on the number of hidden layers. For example, the neural network may include at least 5 layers, at least 10 layers, at least 15 layers, at least 20 layers, at least 25 layers, at least 30 layers, at least 40 layers, at least 50 layers, or at least 100 layers. The nodes of a neural network serve as points of connectivity between adjacent layers. Nodes in adjacent layers form connections with each other, but nodes within the same layer do not form connections with each other. A neural network may include an input layer, n hidden layers, and an output layer. Each layer may comprise a number of nodes.

The system may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 90 Million Gallery, 2015), each of the aforementioned references are incorporated by reference.

Deep learning (also known as deep structured learning, hierarchical learning or deep machine learning) is a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower-level features to form a hierarchical representation. Those features are preferably represented within nodes as feature vectors.

Deep learning by a neural network may include learning multiple levels of representations that correspond to different levels of abstraction; the levels form a hierarchy of concepts. In most preferred embodiments, the neural network includes at least 5 and preferably more than 10 hidden layers. The many layers between the input and the output allow the system to operate via multiple processing layers.

Deep learning is part of a broader family of machine learning methods based on learning representations of data. Neural data can be represented in many ways, e.g., as time series, by frequency, and/or in the spectral and/or time-frequency domains. The familiarity classifier can extract features from this data, which are represented at nodes in the network. Preferably, each feature is structured as a feature vector, a multi-dimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

The systems and methods of the disclosure may use convolutional neural networks (CNN) as part of the familiarity classifier. A CNN is a feedforward network comprising multiple layers to infer an output from an input. CNNs are used to aggregate local information to provide a global predication. CNNs use multiple convolutional sheets from which the network learns and extracts feature maps using filters between the input and output layers. The layers in a CNN connect at only specific locations with a previous layer. Not all neurons in a CNN connect. CNNs may comprise pooling layers that scale down or reduce the dimensionality of features. CNNs hierarchically deconstruct data into general, low-level cues, which are aggregated to form higher-order relationships to identify features of interest. CNNs predictive utility is in learning repetitive features that occur throughout a data set.

The systems and methods of the disclosure may use fully convolutional networks (FCN). In contrast to CNNs, FCNs can learn representations locally within a data set, and therefore, can detect features that may occur sparsely within a data set.

The systems and methods of the disclosure may use recurrent neural networks (RNN). RNNs have an advantage over CNNs and FCNs in that they can store and learn from inputs over multiple time periods and process the inputs sequentially.

The systems and methods of the disclosure may use generative adversarial networks (GAN), which find particular application in training neural networks. One network is fed training exemplars from which it produces synthetic data. The second network evaluates the agreement between the synthetic data and the original data. This allows GANs to improve the prediction model of the second network.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### REFERENCES

Accardo, A., Affinito, M., Carrozzi, M., & Bouquet, F. (1997). Use of the fractal dimension for the analysis of electroencephalographic time series. Biological Cybernetics, 77(5), 339-350.
Ahmadlou, M., Adeli, H., & Adeli, A. (2011). Fractality and a Wavelet-chaos-Methodology for EEG-based Diagnosis of Alzheimer Disease. Alzheimer Disease & Associated Disorders, 25(1), 85-92.
Akar, S. A., Kara, S., Agambayev, S., & Bilgig, V. (2015). Nonlinear analysis of EEGs of patients with major depression during different emotional states. Computers in Biology and Medicine, 67, 49-60.
Akar, S. A., Kara, S., Latifoglu, F., & Bilgig, V. (2015). Investigation of the noise effect on fractal dimension of EEG in schizophrenia patients using wavelet and SSA-based approaches. Biomedical Signal Processing and Control, 18, 42-48.
Al-Nuaimi, A. H. H., Jammeh, E., Sun, L., & Ifeachor, E. (2017). Higuchi fractal dimension of the electroencephalogram as a biomarker for early detection of Alzheimer's disease. 2017 39th Annual International Conference of the IEEE Engineering in Medicine and Biology Society (EMBC), 2320-2324.
Al-Nuaimi, A. H. H., Jammeh, E., Sun, L., & Ifeachor, E. (2018). Complexity Measures for Quantifying Changes in Electroencephalogram in Alzheimer's Disease. Complexity, 2018, 1-12.
Amezquita-Sanchez, J. P., Mammone, N., Morabito, F. C., Marino, S., & Adeli, H. (2019). A novel methodology for automated differential diagnosis of mild cognitive impairment and the Alzheimer's disease using EEG signals. Journal of Neuroscience Methods, 322, 88-95.
Arevalo-Rodriguez, I., Smailagic, N., Figuls, M. R. i, Ciapponi, A., Sanchez-Perez, E., Giannakou, A., Pedraza, O. L., Cosp, X. B., & Cullum, S. (2015). Mini-Mental State Examination (MMSE) for the detection of Alzheimer's disease and other dementias in people with mild cognitive impairment (MCI). Cochrane Database of Systematic Reviews, 3.
Bachmann, M., Paeske, L., Kalev, K., Aarma, K., Lehtmets, A., Ööpik, P., Lass, J., & Hinrikus, H. (2018). Methods for classifying depression in single channel EEG using linear and nonlinear signal analysis. Computer Methods and Programs in Biomedicine, 155, 11-17.
Bahrami, B., Seyedsadjadi, R., Babadi, B., & Noroozian, M. (2005). Brain complexity increases in mania. NeuroReport, 16(2), 187-191.
Bergeron, D., Flynn, K., Verret, L., Poulin, S., Bouchard, R. W., Bocti, C., Fülöp, T., Lacombe, G., Gauthier, S., Nasreddine, Z., & Laforce, R. J. (2017). Multicenter Validation of an MMSE-MoCA Conversion Table. Journal of the American Geriatrics Society, 65(5), 1067-1072.
Cǔkić, M., Stokić, M., Radenković, S., Ljubisavljević, M., Simić, S., & Savić, D. (2020). Nonlinear analysis of EEG complexity in episode and remission phase of recurrent depression. International Journal of Methods in Psychiatric Research, 29(2), e1816.
Cummings, J. L. (2004). Alzheimer's Disease. New England Journal of Medicine, 351(1), 56-67.
Dauwels, J., Srinivasan, K., Ramasubba Reddy, M., Musha, T., Vialatte, F.-B., Latchoumane, C., Jeong, J., & Cichocki, A. (2011). Slowing and Loss of Complexity in Alzheimer's EEG: Two Sides of the Same Coin? International Journal of Alzheimer's Disease, 2011, 539621.
Doyle, T. L. A., Dugan, E. L., Humphries, B., & Newton, R. U. (2004). Discriminating between elderly and young using a fractal dimension analysis of centre of pressure. International Journal of Medical Sciences, 1(1), 11-20.
Fernindez, A., Gómez, C., Hornero, R., & López-Ibor, J. J. (2013). Complexity and schizophrenia. Progress in Neuro-Psychopharmacology and Biological Psychiatry, 45, 267-276.
Folstein, M. F., Folstein, S. E., & McHugh, P. R. (1975). “Mini-mental state”. A practical method for grading the cognitive state of patients for the clinician. Journal of Psychiatric Research, 12(3), 189-198.
Gómez, C., & Hornero, R. (2010). Entropy and Complexity Analyses in Alzheimer's Disease: An MEG Study. The Open Biomedical Engineering Journal, 4(1), 223-235.
Gómez, C., Mediavilla, Á., Hornero, R., Abásolo, D., & Fernández, A. (2009). Use of the Higuchi's fractal dimension for the analysis of MEG recordings from Alzheimer's disease patients. Medical Engineering & Physics, 31(3), 306-313.
Goshvarpour, A., & Goshvarpour, A. (2020). Schizophrenia diagnosis using innovative EEG feature-level fusion schemes. Physical and Engineering Sciences in Medicine, 43(1), 227-238.
Hanseeuw, B. J., Betensky, R. A., Jacobs, H. I. L., Schultz, A. P., Sepulcre, J., Becker, J. A., Cosio, D. M. O., Farrell, M., Quiroz, Y. T., Mormino, E. C., Buckley, R. F., Papp, K. V., Amariglio, R. A., Dewachter, I., Ivanoiu, A., Huijbers, W., Hedden, T., Marshall, G. A., Chhatwal, J. P., . . . Johnson, K. (2019). Association of Amyloid and Tau With Cognition in Preclinical Alzheimer Disease: A Longitudinal Study. JAMA Neurology, 76(8), 915-924.
Hebert, L. E., Beckett, L. A., Scherr, P. A., & Evans, D. A. (2001). Annual Incidence of Alzheimer Disease in the United States Projected to the Years 2000 Through 2050. Alzheimer Disease & Associated Disorders, 15(4), 169-173.
Higuchi, T. (1988). Approach to an irregular time series on the basis of the fractal theory. Physica D: Nonlinear Phenomena, 31(2), 277-283.
Jacob, J. E., & Gopakumar, K. (2018). Automated Diagnosis of Encephalopathy Using Fractal Dimensions of EEG Sub-Bands. 2018 IEEE Recent Advances in Intelligent Computational Systems (RAICS), 94-97.
Langa, K. M., & Levine, D. A. (2014). The Diagnosis and Management of Mild Cognitive Impairment: A Clinical Review. JAMA, 312(23), 2551-2561.
Lau, Z. J., Pham, T., Chen, S. H. A., & Makowski, D. (2022). Brain entropy, fractal dimensions and predictability: A review of complexity measures for EEG in healthy and neuropsychiatric populations. European Journal of Neuroscience, 56Lauterborn, J. C., Scaduto, P., Cox, C. D., Schulmann, A., Lynch, G., Gall, C. M., Keene, C. D., & Limon, A. (2021). Increased excitatory to inhibitory synaptic ratio in parietal cortex samples from individuals with Alzheimer's disease. Nature Communications, 12(1), Article 1.
McKhann, G. M., Knopman, D. S., Chertkow, H., Hyman, B. T., Jack, C. R., Kawas, C. H., Klunk, W. E., Koroshetz, W. J., Manly, J. J., Mayeux, R., Mohs, R. C., Morris, J. C., Rossor, M. N., Scheltens, P., Carrillo, M. C., Thies, B., Weintraub, S., & Phelps, C. H. (2011). The diagnosis of dementia due to Alzheimer's disease: Recommendations from the National Institute on Aging-Alzheimer's Association workgroups on diagnostic guidelines for Alzheimer's disease. Alzheimer's & Dementia, 7(3), 263-269.
Mensen, A., & Khatami, R. (2013). Advanced EEG analysis using threshold-free cluster-enhancement and non-parametric statistics. NeuroImage, 67, 111-118.
Mohammadi, M. R., Khaleghi, A., Nasrabadi, A. M., Rafieivand, S., Begol, M., & Zarafshan, H. (2016). EEG classification of ADHD and normal children using non-linear features and neural network. Biomedical Engineering Letters, 6(2), 66-73.
Nasreddine, Z. S., Phillips, N. A., Bédirian, V., Charbonneau, S., Whitehead, V., Collin, I., Cummings, J. L., & Chertkow, H. (2005). The Montreal Cognitive Assessment, MoCA: A Brief Screening Tool For Mild Cognitive Impairment. Journal of the American Geriatrics Society, 53(4), 695-699.
Nobukawa, S., Yamanishi, T., Nishimura, H., Wada, Y., Kikuchi, M., & Takahashi, T. (2019). Atypical temporal-scale-specific fractal changes in Alzheimer's disease EEG and their relevance to cognitive decline. Cognitive Neurodynamics, 13(1), 1-11.
Puri, D. V., Nalbalwar, S., Nandgaonkar, A., & Wagh, A. (2022). Alzheimer's disease detection from optimal EEG channels and Tunable Q-Wavelet Transform. Indonesian Journal of Electrical Engineering and Computer Science, 25(3), 1420.
Raghavendra, B. S., Dutt, D. N., Halahalli, H. N., & John, J. P. (2009). Complexity analysis of EEG in patients with schizophrenia using fractal dimension. Physiological Measurement, 30(8), 795-808.
Rajan, K. B., Weuve, J., Barnes, L. L., McAninch, E. A., Wilson, R. S., & Evans, D. A. (2021). Population estimate of people with clinical Alzheimer's disease and mild cognitive impairment in the United States (2020-2060). Alzheimer's & Dementia, 17(12), 1966-1975.
Ranasinghe, K. G., Verma, P., Cai, C., Xie, X., Kudo, K., Gao, X., Lerner, H. M., Mizuiri, D., Strom, A., Iaccarino, L., La Joie, R., Miller, B. L., Tempini, M. L. G., Rankin, K. P., Jagust, W. J., Vossel, K. A., Rabinovici, G. D., Raj, A., & Nagarajan, S. S. (2021). Abnormal neural oscillations depicting excitatory-inhibitory imbalance are distinctly associated with amyloid and tau depositions in Alzheimer's disease. Alzheimer's & Dementia, 17(S4).
Reisberg, B., Prichep, L., Mosconi, L., John, E. R., Glodzik-Sobanska, L., Boksay, I., Monteiro, I., Torossian, C., Vedvyas, A., Ashraf, N., Jamil, I. A., & de Leon, M. J. (2008). The pre-mild cognitive impairment, subjective cognitive impairment stage of Alzheimer's disease. Alzheimer's & Dementia, 4(1, Supplement 1), S98-S108.
Rossini, P. M., Di Iorio, R., Vecchio, F., Anfossi, M., Babiloni, C., Bozzali, M., Bruni, A. C., Cappa, S. F., Escudero, J., Fraga, F. J., Giannakopoulos, P., Guntekin, B., Logroscino, G., Marra, C., Miraglia, F., Panza, F., Tecchio, F., Pascual-Leone, A., & Dubois, B. (2020). Early diagnosis of Alzheimer's disease: The role of biomarkers including advanced EEG signal analysis. Report from the IFCN-sponsored panel of experts. Clinical Neurophysiology, 131(6), 1287-1310.
Sabeti, M., Katebi, S., & Boostani, R. (2009). Entropy and complexity measures for EEG signal classification of schizophrenic and control participants. Artificial Intelligence in Medicine, 47(3), 263-274.
Schmand, B., Eikelenboom, P., van Gool, W. A., & Initiative, the A. D. N. (2011). Value of Neuropsychological Tests, Neuroimaging, and Biomarkers for Diagnosing Alzheimer's Disease in Younger and Older Age Cohorts. Journal of the American Geriatrics Society, 59(9), 1705-1710.
Smith, S. M., & Nichols, T. E. (2009). Threshold-free cluster enhancement: Addressing problems of smoothing, threshold dependence and localisation in cluster inference. NeuroImage, 44(1), 83-98.
Smits, F. M., Porcaro, C., Cottone, C., Cancelli, A., Rossini, P. M., & Tecchio, F. (2016). Electroencephalographic Fractal Dimension in Healthy Ageing and Alzheimer's Disease. PLOS ONE, 11(2), e0149587.
Staudinger, T., & Polikar, R. (2011). Analysis of complexity based EEG features for the diagnosis of Alzheimer's disease. 2011 Annual International Conference of the IEEE Engineering in Medicine and Biology Society, 2033-2036.
Sun, J., Wang, B., Niu, Y., Tan, Y., Fan, C., Zhang, N., Xue, J., Wei, J., & Xiang, J. (2020). Complexity Analysis of EEG, MEG, and fMRI in Mild Cognitive Impairment and Alzheimer's Disease: A Review. Entropy, 22(2), Article 2.
Taylor, C. A., Greenlund, S. F., McGuire, L. C., Lu, H., & Croft, J. B. (2017). Deaths from Alzheimer's Disease—United States, 1999-2014. Morbidity and Mortality Weekly Report, 66(20), 521-526.
Tjur, T. (2009). Coefficients of Determination in Logistic Regression Models—A New Proposal: The Coefficient of Discrimination. The American Statistician, 63(4), 366-372.
Vaz, M., & Silvestre, S. (2020). Alzheimer's disease: Recent treatment strategies. European Journal of Pharmacology, 887, 173554.
Wajnsztejn, R., Carvalho, T. D. de, Garner*, D. M., Raimundo, R. D., Vanderlei, L. C. M., Godoy, M. F., Ferreira, C., Valenti, V. E., & Abreu, L. C. de. (2016). Higuchi fractal dimension applied to RR intervals in children with Attention Deficit Hyperactivity Disorder. Journal of Human Growth and Development, 26(2), 147-153.
Wanliss, J. A., & Wanliss, G. E. (2022). Efficient calculation of fractal properties via the Higuchi method. Nonlinear Dynamics, 109(4), 2893-2904.
Yang, A. C., Wang, S.-J., Lai, K.-L., Tsai, C.-F., Yang, C.-H., Hwang, J.-P., Lo, M.-T., Huang, N. E., Peng, C.-K., & Fuh, J.-L. (2013). Cognitive and neuropsychiatric correlates of EEG dynamic complexity in patients with Alzheimer's disease. Progress in Neuro-Psychopharmacology and Biological Psychiatry, 47, 52-61.

1. A system for assessing the cognitive status of a subject, the system comprising:
a central processing unit (CPU); and storage coupled to said CPU for storing instructions that when executed by the CPU cause the CPU to:
accept as an input, neural data from a subject, recorded using EEG sensors, said neural data;
select EEG channels of interest from the neural data transform the neural data of each channel into a time series;
input the time series into a first and second preprocessing stream, wherein:
the first preprocessing stream: (i) performs a wavelet transformation of the time series to yield an amplitude time-course for each EEG channel and frequency of the neural data; (ii) calculates the dominant frequency and highest instantaneous power for each time-point of each time-course to identify momentary artifacts of the recorded neural data in the time series; and
the second preprocessing stream: applies a bandpass filter and/or identifies and interpolates faulty channels; and
remove the identified momentary artifacts from the time series input into the second preprocessing stream to produce preprocessed time series.

2. The system of claim 1, wherein the first preprocessing stream: (i) performs a wavelet transformation of the time series to yield an amplitude time-course for each EEG channel and frequency of the neural data; (ii) calculate a dominant frequency at each time-point of each time-course; (iii) determine the highest instantaneous power at each time-point; (iv) for each EEG channel, provides the dominant frequency and highest instantaneous power to an isolation forest model, thereby training a channel-specific model for each EEG channel that obtain an anomaly score for each time-point; (v) combine the anomaly scores for each time point to produce anomaly score time-courses; subject to the anomaly score time-courses to a threshold using cutoff values for each channel to produce binary vectors; and (vi) use the binary vectors for each channel to identify momentary artifacts in the time series.

3. The system of claim 2, wherein the wavelet transformation is a Morlet wavelet transformation.

4. The system of claim 3, wherein the wavelets span the frequency bands of about 40 Hz to about 100 Hz.

5. The system of claim 1, wherein the momentary artifacts comprise muscle artifacts in the recorded neural data.

6. The system of claim 1, wherein the CPU further:
bandpass filters the preprocessed time series to produce a plurality of different frequency band time series;
repeatedly advance a sliding-window along each frequency band time series and obtain at least one fractal measurement to produce at least one measure time series for each frequency band time series;
extract measures from each measure time series;
combine the extracted measures and analyze the combined measures using a machine learning system trained to correlate features in the combined measures with cognitive status to produce at least one Fractal Dimensions Distribution (FDD) score; and
provide an output of the subject's cognitive status based on the FDD score.

7. The system of claim 6, wherein producing the FDD score comprises determining summary statistics for the distribution of the fractal measurements.

8. The system of claim 7, wherein the summary statistics comprise one or more of a standard deviation of the fractal measurements, mean of the fractal measurements, skewness of the fractal measurements, and kurtosis of the fractal measurements.

9. The system of claim 6, wherein the extracted measures comprise one or more of:
frequency and/or time frequency measures;
oscillatory measures;
amplitude modulation measures;
spectral connectivity measures;
network analysis measures;
chaos measures;
complexity measures; and
entropy measures.

10. The system of claim 6, wherein the output comprises a top-level, all cause assessment of the subject.

11. The system of claim 10, wherein the output further comprises Shapely Additive explanation (SHAP) scores of a per-feature basis.

12. The system of claim 11, wherein the SHAP scores are subject to semantic separation to provide semantic-feature grouping SHAP scores.

13. The system of claim 12, wherein the semantic-feature grouping SHAP scores maintain directionality and magnitude consistent with the sum of the individual features and an overall model prediction probability.

14. The system of claim 12, wherein the semantic feature-grouping SHAP scores are bounded with a 0 point marking a change in the directionality of the group influence on the top-level prediction.

15. The system of claim 6, wherein the system allocates a continuous score of the output into one of a plurality of bins, wherein each bin corresponds to a subject's risk of developing a cognitive impairment.

16. The system of claim 1, wherein the neural data is collected from the subject over at least one period of time during which the subject is performing a task.

17. The system of claim 16, wherein the recorded neural data includes event data correlated with performance of the at least one task.

18. The system of claim 6, wherein the fractal measurement is a calculated a Katz fractal dimension (KFD).

19. The system of claim 6, wherein the fractal measurement is a calculated Higuchi fractal dimension (HFD).
